(19) United States
(12) Patent Application Publication
(10) Pub. No.: US 2021/0409386 A1
(43) Pub. Date: Dec. 30, 2021

(54) IoT PCR FOR DISEASE AND VACCINATION DETECTION AND ITS SPREAD MONITORING USING SECURE BLOCKCHAIN DATA PROTOCOL

(71) Applicant: Tarek Al Sultan, Sharq (KW)

(72) Inventor: Tarek Al Sultan, Sharq (KW)

(21) Appl. No.: 17/352,332

(22) Filed: Jun. 20, 2021

Related U.S. Application Data
(60) Provisional application No. 63/079,867, filed on Sep. 17, 2020.
(60) Provisional application No. 63/044,296, filed on Jun. 25, 2020.
(60) Provisional application No. 63/043,785, filed on Jun. 25, 2020.

(51) Int. Cl. H04L 29/06; H04W 4/80; G16Y 10/60; G16Y 20/40

(52) U.S. Cl. CPC H04L 63/0492; G16Y 20/40; G16Y 10/60; H04W 4/80

(57) ABSTRACT

A system allows a data to be automatically uploaded via a Bluetooth interface to an Android or Apple-based smartphone and then wirelessly sent to a secure blockchain powered global network, instantly making the test results available anywhere in the world. The IoT system presented here could become an essential tool for healthcare centers to tackle infectious disease outbreaks identified either by DNA or ribonucleic acid.

### FIELD OF THE INVENTION

COVID-19, ranked as the most severe pandemic in modern time, emerged as a serious public health concern.

### BACKGROUND OF THE INVENTION

COVID-19, ranked as the most severe pandemic in modern time, emerged as a serious public health concern. Due to the lack of effective treatment of COVID19, accurate and early detection methods to identify the COVID19 serotype are required in order to provide careful patient monitoring and to prevent the disease's progression to a more severe stage. An easy-to-use, affordable, truthful, and reliable point of care (POC) device to perform rapid and economical COVID diagnostic tests in high-risk areas is badly needed. Moreover, it is of the utmost importance to immediately report the results of the POC, including its time and location, to authorities in a centralized location to take required measures. In addition, Global Response Aid are launching a mobile application focusing on managing and storing diagnostic data enabling travel passes.

These days, a vast amount of the human population has possession of a cell phone with an embedded global positioning system (GPS) connected to mobile networks via base stations using different generations of cellular mobile communications, such as second (global system for mobile communications) (2G), third (universal mobile telecommunications system) or fourth (long term evolution (LTE)/worldwide interoperability for microwave access). Connecting the easy to use and cost effective POC devices providing the COVID diagnoses via a mobile network would create an Internet of Things (IoT) or healthcare, an essential tool to tackle any infectious disease outbreak. The IoT would speed up the information transfer from POC devices to a centralized location and then based on models using big data analysis and blockchain for secure transfer, suggest targeting specific sites. The IoT would be able to communicate with the POC systems, inform its operator where to go next to perform testing, remotely change the test protocols or provide other important information to the POC device user.

This year, the fifth generation of cellular mobile communications will be released with a massively increased data rate compared to those of previous generations. Nevertheless, the IoT for POC applications such as COVID testing can rely on the oldest generation of 2G, as the POC communication with the centralized location requires a low data rate, an important feature for cash strapped countries of the developing world.

COVID is often detected by a immunochromatographic test either alone or in combination with enzyme linked immunosorbent assay (ELISA), and/or reverse transcription polymerase chain reaction (RT-PCR) (allowing identification of the COVID serotype). The lateral flow immunochromatographic test is fast and easy to use, therefore is often used in remote areas. However, due to its lower sensitivity as well as specificity, a confirmation using a second detection method (ELISA and/or RT-PCR) is often required. The ELISA method relies on antibody production by the human immune system, the method being more suitable for advanced stages of the disease. The RT-PCR is the preferred method as, in principle, it can detect a single copy of specific ribonucleic acid (RNA) having both sensitivity and specificity required for early disease detection (lowering the cost of treatment).

Unfortunately, RT-PCR tests are typically only carried out in hospitals or certified diagnostic laboratories after the onset of COVID symptoms. The RT-PCR method is highly specific and reliable; the problem is that not every infected patient makes it to the hospital to be positively diagnosed. As an outcome, the diagnostic results are not available as quickly and comprehensively as required for disease outbreak control.

Built on over ten years of real-time expertise, the Applied Biosystems® 7500. Fast and 7500 Real-Time PCR Systems are versatile platforms for the detection and quantification of nucleic acids in standard 96-well formats. Our integrated real-time PCR solutions combine innovative thermal cycling systems, powerful software, optimized reagents, your choice of off-the-shelf or custom assays, and superior support for a variety of applications.

### SUMMARY OF THE INVENTION

During infectious disease outbreaks, the centers for disease control need to monitor particular areas. Considerable effort has been invested in the development of portable, user-friendly, and cost-effective systems for point-of-care (POC) diagnostics, which could also create an Internet of Things (IoT) for healthcare via a global network. However, at present IoT based on a functional POC instrument is not available. Here we show a fast, user-friendly, and affordable IoT system based on a polymerase chain reaction device. We demonstrate below the system's capability by amplification of COVID-19. The resulting data is then automatically uploaded via a Bluetooth interface to an Android or Apple-based smartphone and then wirelessly sent to a secure blockchain powered global network, instantly making the test results available anywhere in the world. The IoT system presented here could become an essential tool for healthcare centers to tackle infectious disease outbreaks identified either by DNA or ribonucleic acid.

### BRIEF DESCRIPTION OF THE DRAWING

Other advantages of the present invention will be readily appreciated as the same becomes better understood by reference to the following detailed description when considered in connection with the accompanying drawings wherein:

FIGS. 1 and 2 show several schematic diagrams demonstrate the IoT system that could be applied for COVID spread monitoring.

### DETAILED DESCRIPTION OF THE INVENTION

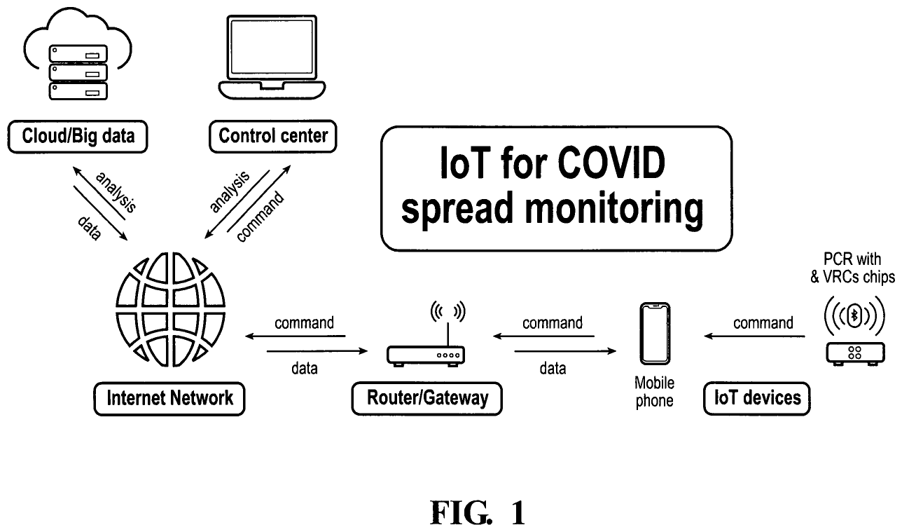

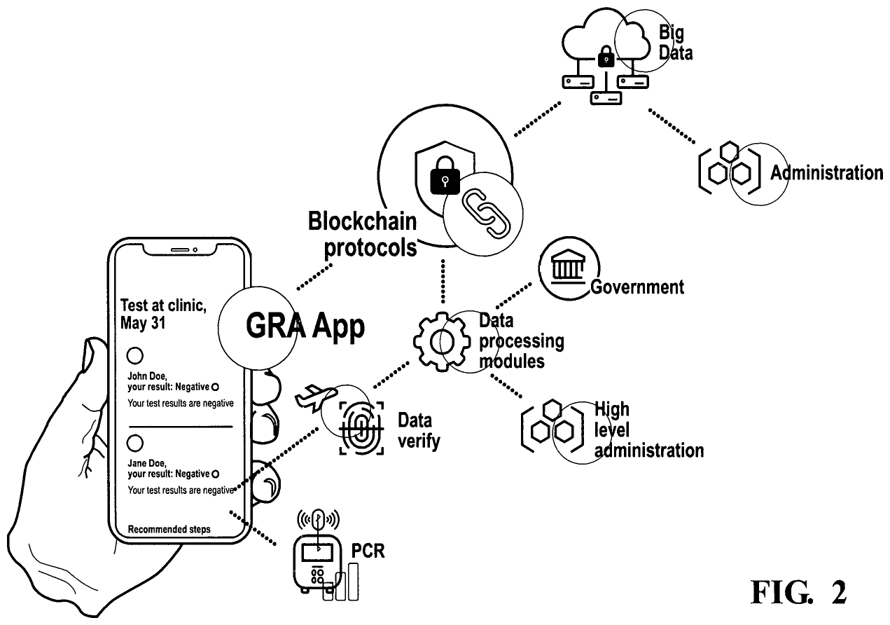

Referring to FIG. 1, a schematic diagram demonstrates a IoT system that could be applied for COVID spread monitoring. Once the sample potentially containing the COVID is processed at point of interest location, the results of diagnoses as well as GPS coordinates of the location are automatically transferred via the user's mobile phone interface through a global network to a control center. All results can be collected as cloud data by a network to create a disease outbreak map showing the disease outbreak area and carry out continuous monitoring.

2. Material and Methods/PCR Device.

2.1. PCR Chip: Fabrication and Assembly.

The core of the system consists of four virtual reaction chambers (VRC) on a hydrophobically coated glass which were successfully tested earlier for ultrafast real-time PCR with external optics, and a fully integrated real-time PCR. The glass will be placed on a silicon chip made by micro-electro-mechanical system (MEMS) technology developed earlier. The layout of the MEMS chip containing heaters and sensors using a Nanolithography toolbox and adding an electrically grounded guard ring between the sensor and the heater. Additionally, the chip size will increase to assure easier handling. The chip will be placed on its own printed circuit board (PCB) and will be connected to the motherboard of the PCR unit by a connector for simple replacement and calibration. The electronics as well as the optics will also be improved but it is not a subject of this contribution. The PCR systems will then be assembled and calibration ready for deployment.

2.2. Bluetooth Module Design.

Wireless communication between mobile platforms, such as a mobile phone or tablet, and the PCR system is provided via a BT module, which comprised a commercial unit supporting BT version 2.1. This unit has its own dedicated 5 V power supply with a universal serial bus (USB) connector, including AC-DC converter and voltage stabilizer, making it compatible with a standard USB power supply or power bank. The independent power supply for the BT module eliminates its influence on PCR system stability.

PCR system is conducted bi-directionally via a universal asynchronous receiver-transmitter interface. Based on a request from a mobile system, such as a smartphone or tablet, the last PCR measurement data are sent to the mobile system.

2.3. Cell Phone/PC APP.

Global Response Aid have developed an application (APP) for Apple and Android devices that allows users to receive data via the BT communication module from the PCR system, save them and represent them in graphical format. The system has the function of sending the data via blockchain to a dedicated place with GPS coordinates to inform authorities about the presence of the infectious disease. After pairing the device with the BT unit of the PCR system, data from the PCR system are downloaded into the device and plotted on its display. They can also be automatically sent via LTE network to a dedicated place monitoring the disease outbreak. For convenience, we also created a personal computer (PC) APP for PCR system programming either via USB or BT interface.

The system of the present invention also offers a secured, blockchain-based human identity stored on one or more blocks in a blockchain. For example, information stored can include a user's medical records or vaccination history and allows a user to visit a medical clinic or medical aid station that has an account in the blockchain-based identity and transaction platform, enter contact information and/or a username, and provide a thumbprint or biometric data to allow the medical clinic to have access to the user's health records and other information included in the blockchain and alert about dates and times of upcoming vaccination.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof.

Therefore, it is intended that the invention not be limited to the particular embodiment disclosed as the best mode contemplated for carrying out this invention. It will be apparent that multiple embodiments of this disclosure may be practiced without some or all of these specific details. In other instances, well-known process operations have not been described in detail in order not to unnecessarily obscure the present embodiments.

1. A spread of disease monitoring. system comprising;
a first device adaptable to allow a data to be automatically uploaded via a Bluetooth interface to an Android or Apple-based smartphone; and
a second device adaptable for wirelessly sending the data to a secure blockchain powered global network, instantly making the test results available anywhere in the world.
